(19) United States
(12) Patent Application Publication
(10) Pub. No.: US 2015/0152130 A1
(43) Pub. Date: Jun. 4, 2015

(54) TRANS-RESVERATROL POLYSACCHARIDE, METHOD FOR PRODUCING THE SAME, AND COMPOSITION COMPRISING THE SAME

(71) Applicant: FAITH, INC., Osaka (JP)

(72) Inventors: Hiroki HAMADA, Okayama (JP); Shinichi KOJIMA, Osaka (JP); Shoji TAKAHASHI, Osaka (JP); Chiemi MOMOTA, Osaka (JP)

(21) Appl. No.: 14/592,702

(22) Filed: Jan. 8, 2015

Related U.S. Application Data
(63) Continuation-in-part of application No. 14/329,249, filed on Jul. 11, 2014.

(30) Foreign Application Priority Data
Oct. 8, 2013 (JP) 2013-211126

(51) Int. Cl. C07H 15/203; A61Q 19/00; C12P 19/44; A61K 8/60

(52) U.S. Cl. CPC C07H 15/203; A61K 8/602; A61Q 19/00; C12P 19/44; A61K 2800/10

(57) ABSTRACT

An object of the present invention is to provide a trans-resveratrol derivative that resists isomerization to the cis-form. Another object of the present invention is to provide a trans-resveratrol derivative that has no toxicity against cells and has sufficient antioxidative properties and/or a sufficient whitening effect. This object can be achieved by a method for producing a trans-resveratrol polysaccharide, the method comprising the step of bringing a trans-resveratrol glucoside into contact with sugar in the presence of γ-cyclodextrin glucanotransferase.

### TECHNICAL FIELD

The present invention relates to trans-resveratrol polysaccharide, a method for producing the same, a liposome comprising the same, and a composition comprising the same.

### BACKGROUND ART

It is widely known that resveratrol exhibits antioxidative properties, such as protection from cell death induced by hydrogen peroxide, etc., in the body. However, resveratrol is poorly soluble in water, and monosaccharide glycosides of resveratrol, which have improved water solubility, are known.

Further, as shown in PTL 1, a technique using cyclodextrin glucanotransferase has been developed as a technique for producing polysaccharides of polyphenols.

Moreover, it has been revealed that trans-resveratrol has the effects of inducing NFκB activation, etc., but cis-resveratrol does not have such effects (NPL 1).

### CITATION LIST

### Patent Literature

PTL 1: JP5124712B

### Non-Patent Literature

NPL 1: J Immunol, 2010 Sep. 15; 185(6): 3718-27

### SUMMARY OF INVENTION

### Technical Problem

The present inventors found that monosaccharide glycosides of trans-resveratrol underwent cis-trans isomerization, thereby resulting in the isomerization of about 15% of the trans-form to the cis-form.

Moreover, as shown in NPL 1, since cis-resveratrol is considered not to exhibit antioxidative properties, etc., in the body, there is demand for the production of trans-resveratrol derivatives that are stable as the trans-form, and that are not isomerized to the cis-form upon stimulation, such as heat, a pH environment, or ultraviolet radiation, when they are mixed into food-and-drink products or cosmetics.

PTL 1 discloses a method for producing polysaccharides of polyphenols, such as curcumine and flavanol, but does not disclose the production of resveratrol polysaccharides. In fact, PTL 1 does not disclose any techniques relating to the production of polysaccharides of polyphenol compounds having a stilbene skeleton, which is the base of cis-trans isomerization.

Moreover, trans-resveratrol or monosaccharide glycoside thereof is used as an active ingredient of various compositions; however, cytotoxicity caused by trans-resveratrol or monosaccharide glycoside thereof has not been sufficiently examined, and the antioxidative properties of trans-resveratrol or monosaccharide glycoside thereof are not sufficient.

Furthermore, the whitening effect of trans-resveratrol or monosaccharide glycoside thereof has been suggested, but this effect is not sufficient to satisfy market demands.

In view of the above background art, an object of the present invention is to provide a trans-resveratrol derivative that resists isomerization to the cis-form. Another object of the present invention is to provide a trans-resveratrol derivative that has low toxicity against cells and has excellent antioxidative properties and/or an excellent whitening effect.

### Solution to Problem

As a result of conducting extensive research to solve the above problems, the present inventors succeeded in producing a trans-resveratrol polysaccharide suitable for the above object by using a specific resveratrol glycoside as a starting material and using a specific enzyme.

The present invention has been completed on the basis of this finding and broadly includes inventions according to the following embodiments.

Item 1. A method for producing a trans-resveratrol polysaccharide, the method comprising the step of bringing a trans-resveratrol glucoside into contact with sugar in the presence of γ-cyclodextrin glucanotransferase.
Item 2. The production method according to Item 1, wherein the trans-resveratrol glucoside is at least one selected from the group consisting of trans-resveratrol-3-O-β-D-monoglucoside, trans-resveratrol-4′-O-β-D-monoglucoside, and trans-resveratrol-2,3′-O-β-D-bis-diglucoside.
Item 3. The production method according to Item 1 or 2, wherein the sugar is at least one monosaccharide selected from the group consisting of glucose, galactose, mannose, xylose, fructose, rhamnose, arabinose, allose, altrose, idose, N-acetylglucosamine, N-acetylgalactosamine, talose, glucuronic acid, glucosamine, galactosamine, and fucose; or a sugar in which two or more of the same or different members of these monosaccharides are linked together.
Item 4. The production method according to Item 3, wherein the sugar is a sugar in which two or more monosaccharides are linked together, and the sugar has a terminal glucose residue.
Item 5. The production method according to any one of Items 1 to 4, wherein the sugar is at least one member selected from the group consisting of α-cyclodextrin, β-cyclodextrin, and γ-cyclodextrin.
Item 6. The production method according to any one of Items 1 to 5, wherein the γ-cyclodextrin glucanotransferase is derived from at least one bacterium selected from the group consisting of bacteria belonging to the genus Bacillus, bacteria belonging to the genus Brevibacterium, bacteria belonging to the genus Klebsiella, and bacteria belonging to the genus Corynebacterium.
Item 7. At least one trans-resveratrol polysaccharide selected from the group consisting of:

trans-resveratrol-3-O-β-D-diglucoside, and compounds in which sugar is further linked to the hydroxyl group at the 2G-4 position of trans-resveratrol-3-O-β-D-diglucoside via glycoside linkage;

trans-resveratrol-4′-O-β-D-diglucoside, and compounds in which sugar is further linked to the hydroxyl group at the 2G-4 position of trans-resveratrol-4′-O-β-D-diglucoside via glycoside linkage;
trans-resveratrol-O-β-D-3-diglucoside-4′-monoglycoside, and compounds in which sugar is further linked to the hydroxyl group at the 2G-4 position of trans-resveratrol-O-β-D-3-diglucoside-4′-monoglycoside via glycoside linkage;

trans-resveratrol-O-β-D-3-monoglucoside-4′-diglycoside, and compounds in which sugar is further linked to the hydroxyl group at the 2G′-4 position of trans-resveratrol-O-β-D-3-monoglucoside-4′-diglycoside via glycoside linkage; and

trans-resveratrol-O-β-D-3-diglucoside-4′-diglycoside, and compounds in which sugar is further linked to the hydroxyl group at the 2G-4 position and/or the 2G′-4 position of trans-resveratrol-O-β-D-3-diglucoside-4′-diglycoside via glycoside linkage.

Item 8. The trans-resveratrol polysaccharide according to Item 7, wherein the sugar is at least one monosaccharide selected from the group consisting of glucose, galactose, mannose, xylose, fructose, rhamnose, arabinose, allose, altrose, idose, N-acetylglucosamine, N-acetylgalactosamine, talose, glucuronic acid, glucosamine, galactosamine, and fucose; or a sugar in which two or more of the same or different members of these monosaccharides are linked together.
Item 9. The trans-resveratrol polysaccharide according to Item 7 or 8, wherein the trans-resveratrol polysaccharide has a sugar chain of 2 to 10 linked monosaccharides.
Item 10. The trans-resveratrol polysaccharide according to any one of Items 7 to 9, which is represented by formulae (1) to (3) below:

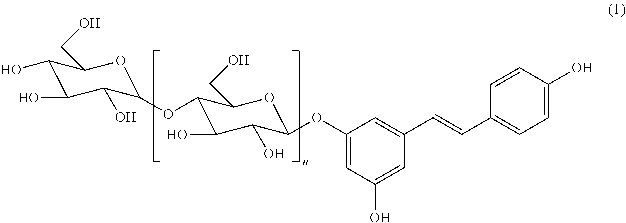

wherein n is an integer of 1 to 9;

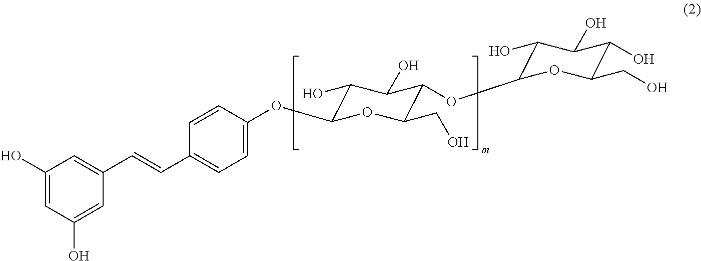

wherein m is an integer of 1 to 9; and

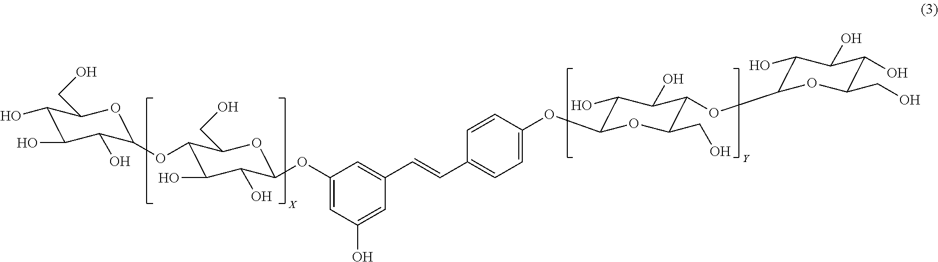

wherein X and Y are each an integer of 1 to 9; and when X is 0, Y is an integer of 1 to 9, and when Y is 0, X is an integer of 1 to 9.
Item 11. A liposome encapsulating the trans-resveratrol polysaccharide according to any one of Items 7 to 10.
Item 12. A composition comprising the trans-resveratrol polysaccharide according to any one of Items 7 to 10 and/or the liposome according to Item 11.
Item 13. The composition according to Item 12, which is a food-and-drink composition or a cosmetic composition.

### Advantageous Effects of Invention

The trans-resveratrol polysaccharide of the present invention is highly water soluble.

The trans-resveratrol polysaccharide of the present invention is easily taken into cells when it is made to act on the body.

The trans-resveratrol polysaccharide of the present invention is not easily isomerized to the cis-form, and is stably maintained as the trans-form.

The trans-resveratrol polysaccharide of the present invention includes one having excellent safety against the body.

The trans-resveratrol polysaccharide of the present invention includes one having excellent rescue activity from cell death.

The trans-resveratrol polysaccharide of the present invention includes one having an excellent effect of suppressing the expression of NFκB, which is a transcription factor that induces inflammation.

The trans-resveratrol polysaccharide of the present invention includes one exhibiting an excellent whitening effect.

### BRIEF DESCRIPTION OF DRAWINGS

FIG. 1 shows column chromatogram by HPLC of a reactant obtained by using trans-resveratrol-3-O-β-D-monoglucoside as a starting material in Production Example 1.

FIG. 2 shows NMR of disaccharide produced by using trans-resveratrol-3-O-β-D-monoglucoside as a starting material in Production Example 1, and the assignment results.

FIG. 3 shows NMR of disaccharide produced by using trans-resveratrol-4′-O-β-D-monoglucoside as a starting material, and the assignment results.

FIG. 4 shows experimental results in which resveratrol derivatives were produced using plant cells. (A) shows the yield of each of the produced derivatives. (B) shows the chemical structure of each of the produced derivatives.

FIG. 5 shows the mass analysis results of the trans-resveratrol polysaccharides (fractions (a) and (b)) shown in Production Example 1.

FIG. 6 shows the mass analysis results of the trans-resveratrol polysaccharides (fractions (c) and (d)) shown in the production example.

FIG. 7 is a photographic image showing the solubility of the trans-resveratrol polysaccharides shown in the production example.

FIG. 8 shows the experimental results that confirm the cis-trans isomerization of the trans-resveratrol polysaccharides shown in the production example.

FIG. 9 is a graph showing the experimental results presented in Example 2 for confirming the safety.

FIG. 10 is a graph showing the experimental results presented in Example 3 for confirming the rescue activity from cell death.

FIG. 11 is a graph showing the experimental results presented in Example 4 for confirming the effect of suppressing the expression of NFκB.

FIG. 12 shows the experimental results presented in Example 5 for confirming the whitening effect. (A) shows photographic images of epithelial tissue models, and (B) is a graph showing their analysis results. The bar in the image indicates a length of 125 μm.

FIG. 13 shows the experimental results presented in Example 6 for confirming the whitening effect. (A) shows photographic images of epithelial tissue models, and (B) is a graph showing their analysis results. The bar in the image indicates a length of 125 μm.

### DESCRIPTION OF EMBODIMENTS

### Method for Producing Trans-Resveratrol Polysaccharide

The method for producing a trans-resveratrol polysaccharide according to the present invention comprises the step of bringing a trans-resveratrol glucoside into contact with sugar in the presence of γ-cyclodextrin glucanotransferase.

Although the trans-resveratrol glucoside used as a starting material in the production method of the present invention is not particularly limited, for example, trans-resveratrol-3-O-β-D-monoglucoside, trans-resveratrol-4′-O-β-D-monoglucoside, trans-resveratrol-3,4′-O-β-D-bis-diglucoside, etc., can be used.

The above trans-resveratrol glucosides can be produced by using a known method. They can also be purchased from, for example, Tokyo Kasei Kogyo Co., Ltd., or isolated from plants.

The sugar used as a starting material in the production method of the present invention may be a monosaccharide or a sugar in which two or more monosaccharides are linked together via glycoside linkage.

The glycoside linkage is not limited to S-glycoside linkage, N-glycoside linkage, or O-glycoside linkage. Moreover, the glycoside linkage may be α-glycoside linkage or β-glycoside linkage, and further may be 1-4 glycoside linkage or 1-6 glycoside linkage.

Specific examples of monosaccharides include, but are not limited to, glucose, galactose, mannose, xylose, fructose, rhamnose, arabinose, allose, altrose, idose, N-acetylglucosamine, N-acetylgalactosamine, talose, glucuronic acid, glucosamine, galactosamine, fucose, and the like.

Among these, glucose, galactose, mannose, xylose, etc., are preferred, and glucose is more preferred, in terms of the efficient production of the trans-resveratrol polysaccharide of the present invention.

When a monosaccharide is used as sugar, glucose is suitably used.

When the sugar is a sugar in which two or more monosaccharides are linked together, the same or different members of the monosaccharides described above may be linked together.

Further, when the sugar is a sugar in which two or more of the above monosaccharides are linked together, it is preferable that the sugar has a terminal glucose residue.

The most preferred examples of the sugar include α-cyclodextrin, β-cyclodextrin, γ-cyclodextrin, and the like.

Although the γ-cyclodextrin glucanotransferase used in the production method of the present invention is not particularly limited, examples include γ-cyclodextrin glucanotransferase derived from bacteria belonging to the genus Bacillus, bacteria belonging to the genus Brevibacterium, bacteria belonging to the genus Klebsiella, bacteria belonging to the genus Corynebacterium, or like bacteria.

Among these, γ-cyclodextrin glucanotransferase derived from bacteria belonging to the genus Bacillus is preferred. Specific examples include γ-cyclodextrin glucanotransferase derived from Bacillus macerans, Bacillus stearothermophilus, Bacillus circulans, Bacillus megaterium, Bacillus polymyxa, or the like; most preferred is γ-cyclodextrin glucanotransferase derived from Bacillus macerans.

γ-Cyclodextrin glucanotransferase can be produced from the bacterial cells mentioned above, or can be easily purchased from, for example, Amano Enzyme Inc. Further, γ-cyclodextrin glucanotransferase can be obtained by transforming a host (e.g., E. coli) with nucleic acid encoding γ-cyclodextrin glucanotransferase, culturing the host and collecting the cultured product, and obtaining γ-cyclodextrin glucanotransferase from the cultured host through isolation, purification, and other processes.

The amounts of the trans-resveratrol glucoside and sugar used in the production method of the present invention may be set so that the molar ratio of glucose residues contained in the sugar to the trans-resveratrol glucoside (glucose residue/trans-resveratrol glucoside) is about 10 to 10,000. When this molar ratio is 10 or more, the sugar is easily decomposed, and catalytic reaction by γ-cyclodextrin glucanotransferase tends to easily proceed. From this point of view, the molar ratio is preferably 100 or more.

Moreover, when the molar ratio is 10,000 or less, decomposition of the sugar easily occurs, and no linkage reaction occurs between the sugars, resulting in a tendency to suppress the production of high-molecular-weight sugar chains. From this point of view, the molar ratio is preferably 2,000 or less.

The amount of γ-cyclodextrin glucanotransferase used in the production method of the present invention is not particularly limited. Generally, the amount is about 300 to 1,000 units, and preferably 400 to 800 units, per μmol of the trans-resveratrol glucoside.

Although the temperature at which a trans-resveratrol glucoside is brought into contact with sugar in the presence of γ-cyclodextrin glucanotransferase in the production method of the present invention is not particularly limited, the temperature is generally about 20° C. or more and less than 70° C., preferably 25° C. or more, more preferably 35° C. or more, and also preferably 60° C. or less, in consideration of the optimum activity of γ-cyclodextrin glucanotransferase, and in terms of avoiding its inactivation.

Although the pH environment in which a trans-resveratrol glucoside is brought into contact with sugar in the presence of γ-cyclodextrin glucanotransferase in the production method of the present invention is not particularly limited, the pH is generally about 3 to 11, and preferably 4 to 8, in terms of the optimum activity of γ-cyclodextrin glucanotransferase.

The time for bringing a trans-resveratrol glucoside into contact with sugar in the presence of γ-cyclodextrin glucanotransferase in the production method of the present invention may be suitably adjusted so that a trans-resveratrol polysaccharide having a desired sugar chain is obtained. Generally, the time is about 1 minute to 100 hours.

In the above method, the longer the contact time is, the lower the number of monosaccharides forming sugar chains of the resulting trans-resveratrol polysaccharide tends to be. For example, when α-cyclodextrin is used as sugar, a trans-resveratrol polysaccharide in which the largest number of monosaccharides is linked together is obtained for a contact time of about 1 minute to 6 hours. When β-cyclodextrin is used as sugar, a trans-resveratrol polysaccharide in which the largest number of monosaccharides is linked together is obtained for a contact time of about 1 to 12 hours. When γ-cyclodextrin is used as sugar, a trans-resveratrol polysaccharide in which the largest number of monosaccharides is linked together is obtained for a contact time of about 3 to 24 hours. The linkage pattern of monosaccharides in the above polysaccharides is not particularly limited.

Although the method of stopping the contact is not particularly limited, for example, a known method, such as heating, may be used to inactivate the enzyme, i.e., γ-cyclodextrin glucanotransferase.

As described above, by the step of bringing a trans-resveratrol glucoside into contact with sugar in the presence of γ-cyclodextrin glucanotransferase based on the production method of the present invention, mixtures containing various resveratrol polysaccharides are obtained, as described in detail in the “trans-Resveratrol Polysaccharide” section below.

In the production method of the present invention, the desired trans-resveratrol polysaccharide can be obtained from such a mixture by isolation, purification, and other processes. For example, a known isolation method and purification method, such as column chromatography, can be used.

### Trans-Resveratrol Polysaccharide

The trans-resveratrol polysaccharide of the present invention is at least one selected from the group consisting of:

trans-resveratrol-3-O-β-D-diglucoside, and compounds in which sugar is further linked to the hydroxyl group at the 2G-4 position of trans-resveratrol-3-O-β-D-diglucoside via glycoside linkage;

trans-resveratrol-4′-O-β-D-diglucoside, and compounds in which sugar is further linked to the hydroxyl group at the 2G-4 position of trans-resveratrol-4′-O-β-D-diglucoside via glycoside linkage;
trans-resveratrol-O-β-D-3-diglucoside-4′-monoglycoside, and compounds in which sugar is further linked to the hydroxyl group at the 2G-4 position of trans-resveratrol-O-β-D-3-diglucoside-4′-monoglycoside via glycoside linkage;

trans-resveratrol-O-β-D-3-monoglucoside-4′-diglycoside, and compounds in which sugar is further linked to the hydroxyl group at the 2G′-4 position of trans-resveratrol-O-β-D-3-monoglucoside-4′-diglycoside via glycoside linkage; and trans-resveratrol-O-β-D-3-diglucoside-4′-diglycoside, and compounds in which sugar is further linked to the hydroxyl group at the 2G-4 position and/or the 2G′-4 position of trans-resveratrol-O-β-D-3-diglucoside-4′-diglycoside via glycoside linkage.

trans-Resveratrol-3-O-β-D-diglucoside is a compound represented by the following formula (4):

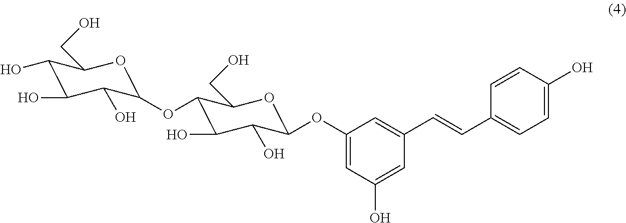 [SMILES: OCC1OC(OC2C(CO)OC(Oc3cc(O)cc(/C=C/c4ccc(O)cc4)c3)C(O)C2O)C(O)C(O)C1O]

The hydroxyl group at the 2G-4 position of trans-resveratrol-3-O-β-D-diglucoside refers to the hydroxyl group at the 4-position of the terminal glucose residue of diglucoside linked to the hydroxyl group at the 3-position of trans-resveratrol via β-glycoside linkage.

trans-Resveratrol-4′-O-β-D-diglucoside is a compound represented by the following formula (5):

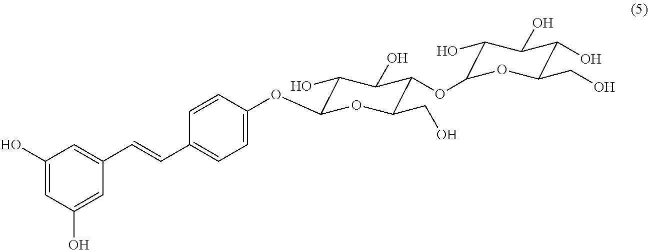 [SMILES: OCC1OC(OC2C(CO)OC(Oc3ccc(/C=C/c4cc(O)cc(O)c4)cc3)C(O)C2O)C(O)C(O)C1O]

The hydroxyl group at the 2G-4 position of trans-resveratrol-4′-O-β-D-diglucoside refers to the hydroxyl group at the 4-position of the terminal glucose residue of diglucoside linked to the hydroxyl group at the 4′-position of trans-resveratrol via β-glycoside linkage.

trans-Resveratrol-O-β-D-3-diglucoside-4′-monoglycoside is a compound represented by the following formula (6):

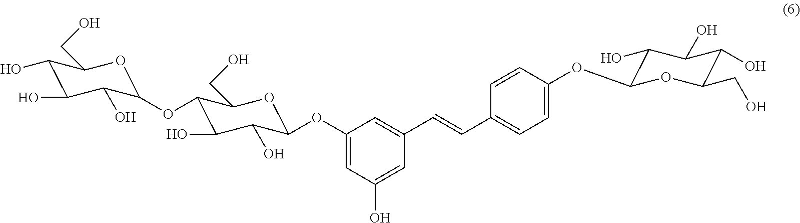 [SMILES: OCC1OC(Oc2ccc(/C=C/c3cc(O)cc(OC4OC(CO)C(OC5OC(CO)C(O)C(O)C5O)C(O)C4O)c3)cc2)C(O)C(O)C1O]

The hydroxyl group at the 2G-4 position of trans-resveratrol-O-β-D-3-diglucoside-4′-monoglycoside refers to the hydroxyl group at the 4-position of the terminal glucose residue of diglucoside linked to the hydroxyl group at the 3-position of trans-resveratrol via β-glycoside linkage.

trans-Resveratrol-O-β-D-3-monoglucoside-4′-diglycoside is a compound represented by the following formula (7):

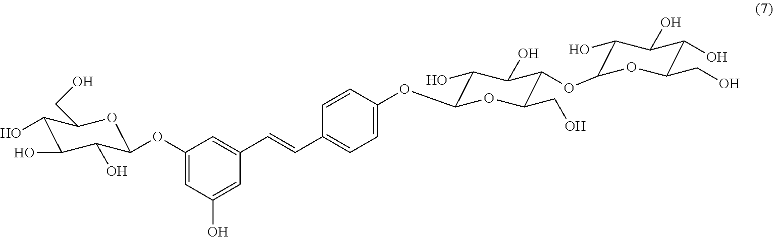 [SMILES: OCC1OC(Oc2cc(O)cc(/C=C/c3ccc(OC4OC(CO)C(OC5OC(CO)C(O)C(O)C5O)C(O)C4O)cc3)c2)C(O)C(O)C1O]

The hydroxyl group at the 2G′-4 position of trans-resveratrol-O-β-D-3-monoglucoside-4′-diglycoside refers to the hydroxyl group at the 4-position of the terminal glucose residue of diglucoside linked to the hydroxyl group at the 4′-position of trans-resveratrol via β-glycoside linkage.

trans-Resveratrol-O-β-D-3-diglucoside-4′-diglycoside is a compound represented by the following formula (8):

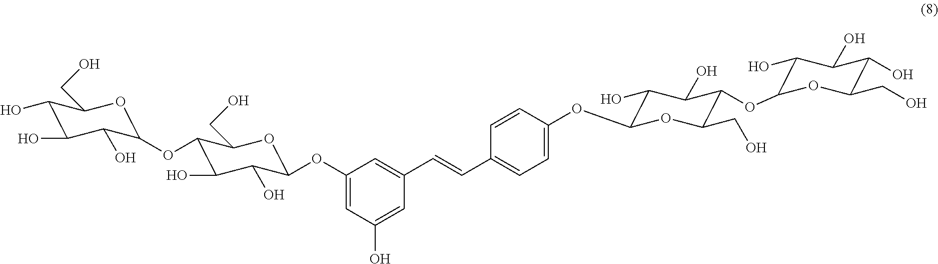 [SMILES: OCC1OC(OC2C(CO)OC(Oc3ccc(/C=C/c4cc(O)cc(OC5OC(CO)C(OC6OC(CO)C(O)C(O)C6O)C(O)C5O)c4)cc3)C(O)C2O)C(O)C(O)C1O]

The hydroxyl group at the 2G-4 position of trans-resveratrol-O-β-D-3-diglucoside-4′-diglycoside refers to the hydroxyl group at the 4-position of the terminal glucose residue of diglucoside linked to the hydroxyl group at the 3-position of trans-resveratrol via β-glycoside linkage.

The hydroxyl group at the 2G′-4 position of trans-resveratrol-O-β-D-3-diglucoside-4′-diglycoside refers to the hydroxyl group at the 4-position of the terminal glucose residue of diglucoside linked to the hydroxyl group at the 4′-position of trans-resveratrol via β-glycoside linkage.

The sugar linked to the hydroxyl group at the 2G-4 position and/or the 2G′-4 position via glycoside linkage may be a monosaccharide or a sugar chain of two or more monosaccharides that are linked together via glycoside linkage.

The glycoside linkage is not limited to S-glycoside linkage, N-glycoside linkage, or O-glycoside linkage. Moreover, the glycoside linkage may be α-glycoside linkage or β-glycoside linkage, and further may be 1-4 glycoside linkage or 1-6 glycoside linkage.

Specific examples of monosaccharides include, but are not limited to, glucose, galactose, mannose, xylose, fructose, rhamnose, arabinose, allose, altrose, idose, N-acetylglucosamine, N-acetylgalactosamine, talose, glucuronic acid, glucosamine, galactosamine, fucose, and the like.

The glycoside linkage between the sugar and the hydroxyl group at the 2G-4 position and/or the 2G′-4 position is not limited to S-glycoside linkage, N-glycoside linkage, or O-glycoside linkage. Moreover, the glycoside linkage may be α-glycoside linkage or β-glycoside linkage, and further may be 1-4 glycoside linkage or 1-6 glycoside linkage.

The trans-resveratrol polysaccharide of the present invention has a sugar chain of linked monosaccharides. The sugar chain generally has 2 to 10 linked monosaccharides; preferably 2 to 9, 2 to 8, or 2 to 7 linked monosaccharides; more preferably 2 to 6, 2 to 5, or 2 to 4 linked monosaccharides; even more preferably 6, 5, 4, or 3 linked monosaccharides; and most preferably 2 linked monosaccharides.

Specific examples of the trans-resveratrol polysaccharide of the present invention include compounds represented by the following chemical formulae (1) to (3):

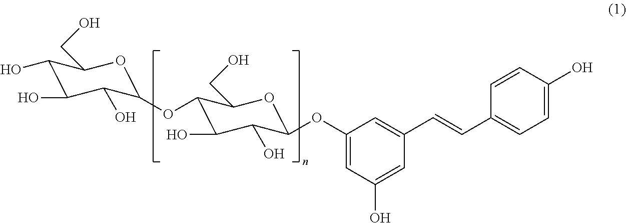

wherein n is an integer of 1 to 9;

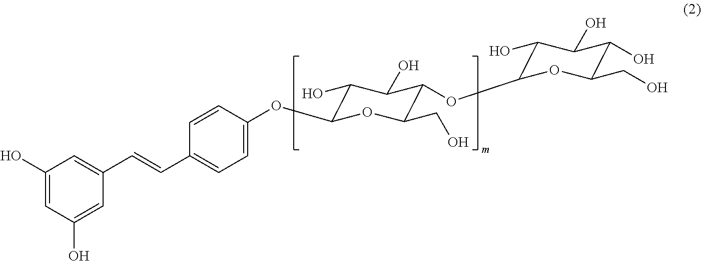

wherein m is an integer of 1 to 9; and

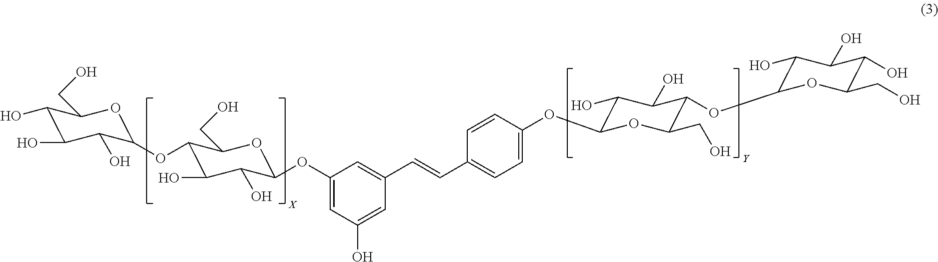

wherein X and Y are each an integer of 1 to 9; and when X is 0, Y is an integer of 1 to 9, and when Y is 0, X is an integer of 1 to 9.

Among the trans-resveratrol polysaccharides of the present invention, trans-resveratrol-O-β-D-4′-diglycoside is preferred in terms of its high in vivo activity. Specifically, this compound is represented by chemical formula (5) above.

The method for producing the trans-resveratrol polysaccharide of the present invention is not particularly limited. For example, the method described above in the “Method for Producing trans-Resveratrol Polysaccharide” section can be appropriately used.

The trans-resveratrol polysaccharide of the present invention tends to resist undergoing cis-trans isomerization. That is, the trans-resveratrol polysaccharide of the present invention resists isomerization to the cis-form upon receiving stimulation, such as heat, pH, or ultraviolet radiation, and is stable as the trans-form.

The trans-resveratrol polysaccharide of the present invention is highly soluble in water, and is easily taken into cells when it is made to act on the body.

Trans-resveratrol that is not modified with sugar chains (hereinafter also referred to as “trans-resveratrol aglycone”) exhibits excellent effects, such as promotion of SOD production, and elimination of singlet oxygen in the body; inhibition of tyrosinase activity, thereby suppressing dopachrome production; inhibition of hyaluronidase activity, collagenase activity, elastase activity, etc.; inhibition of the Maillard reaction; acceleration of the expression of sirtuin gene clusters; suppression of the expression of NFκB, which is a transcription factor that induces inflammation; inhibition of the growth of acne bacteria; and reduction in fat production in the liver, and acceleration of fat degradation in the liver
(Cell Physiol Biochem, 2013; 31(2-3): 230-41,
Biochem Biophys Res Commun, 2003 Aug. 8; 307(4): 861-3,
Biochim Biophys Acta, 2000; 1478(1): 51-60,
Wound Repair Regen, 2013; 21(4): 616-23,
J Antimicrob Chemother, 2007; 59(6): 1182-4,
J Biol Chem, 2012; 287(45): 38050-63,
Mol Cell Biol, 2007; 27(13): 4698-707).

Glycosides and aglycones of vitamin C, vitamin P, quercetin, curcumine, capsaicin, piceatannol, and like compounds are known to exhibit almost the same physiological activity. The reason for this is considered to be that when a glycoside of such a compound is made to act on cells, the glycoside is taken into the cells in the form of aglycone, or the sugar is removed from the glycoside to form an aglycone in the cells.

In particular, when a glycoside of piceatannol having a stilbene skeleton is made to act on cells, the glycoside is considered to act in the form of aglycone in the cells, similar to the manner in which resveratrol does. Therefore, the trans-resveratrol polysaccharide of the present invention is expected to exhibit the same physiological activity as the above trans-resveratrol aglycones.

Further, as shown in the following Examples, the trans-resveratrol polysaccharide of the present invention has less toxicity against the body than trans-resveratrol aglycones.

Moreover, the trans-resveratrol polysaccharide of the present invention has superior rescue activity from cell death induced by hydrogen peroxide, etc., than trans-resveratrol aglycones.

Furthermore, the trans-resveratrol polysaccharide of the present invention itself exhibits a superior whitening effect than trans-resveratrol aglycones.

In consideration of the solubility and stability as the trans-form of the trans-resveratrol polysaccharide of the present invention, which is expected to exhibit the same activity as trans-resveratrol aglycones, compositions comprising the trans-resveratrol polysaccharide of the present invention as an active ingredient are expected to exhibit effects such as anti-aging, skin whitening, skin moisturization, retention of skin elasticity, anti-inflammation, suppression of the development or aggravation of pimples, and prevention of metabolic syndrome. Such compositions can be particularly applied in the field of cosmetics or food-and-drink products.

### Liposome

The liposome of the present invention encapsulates the trans-resveratrol polysaccharide of the present invention described above.

The term “encapsulate” means a state where the trans-resveratrol polysaccharide of the present invention is completely contained in the liposome, or a state where part of the molecules of the trans-resveratrol polysaccharide of the present invention penetrates the lipid bilayer membrane of the liposome. In the specification, the term “enclose” is sometimes used in the same meaning as “encapsulate.”

The amount of the trans-resveratrol polysaccharide encapsulated in the liposome of the present invention is not particularly limited, and is generally about 0.001 to 99.9 wt. % based on 100 wt. % of the liposome of the present invention.

The liposome of the present invention may be neutral, cationic, or anionic, and is not particularly limited.

The liposome of the present invention may be hollow or filled with water or a buffer, such as PBS, MES, ADA, PIPES, ACES, cholamine chloride, TBS, BES, TES, HEPES, citric acid, boric acid, or tartaric acid. Moreover, a close-packed structure comprising the liposome constituent components mentioned below and the trans-resveratrol polysaccharide mentioned above as main components may be formed.

The particle size of the liposome of the present invention is not particularly limited, and can be suitably set within a range that does not impair the effect of the trans-resveratrol polysaccharide of the present invention, for example, when the liposome of the present invention is mixed into a composition. Specifically, the particle size may be about 1 nm to 1 mm. Although the method for measuring the particle size is not particularly limited, for example, a method that measures the particle size as the z-average particle diameter determined by dynamic light scattering can be used.

The structure of the liposome of the present invention may be multilamellar liposome (MLV), large unilamellar liposome (LUV), small unilamellar liposome (SUV), or giant unilamellar liposome (GUV).

The particle size and structure of the liposome of the present invention can be suitably adjusted by using a known method using an extruder, etc.

Examples of the liposome constituent lipids include, but are not limited to, phospholipids, cholesterols, fatty acids, etc.

Specific examples of phospholipids include phosphatidylcholine, phosphatidylserine, phosphatidylglycerol, phosphatidylinositol, phosphatidylethanolamine, phosphatidic acid, cardiolipin, sphingomyelin, egg yolk lecithin, soy lecithin, lysolecithin, and natural phospholipids obtained by hydrogenating the above according to an ordinal method; distearoyl phosphatidylcholine, dipalmitoylphosphatidylcholine (DPPC), dipalmitoylphosphatidylethanolamine (DPPE), dithiodipyridine-dipalmitoylphosphatidylethanolamine (DTP-DPPE), dipalmitoylphosphatidylglycerol (DPPG), dipalmitoylphosphatidylserine (DPPS), eleostearoylphosphatidylcholine, eleostearoylphosphatidylethanolamine, eleostearoylphosphatidylserine, and like synthetic phospholipids; etc.

The phospholipids, cholesterols, and fatty acids described above may be suitably modified. Although the modification is not particularly limited, examples include modification with polyalkylene glycol such as polyethylene glycol or polypropylene glycol, modification with sugar chains, etc. These phospholipids may be suitably used in combination.

Examples of cholesterols include cholesterol, phytosterol, etc. These can be suitably used in combination.

Examples of fatty acids include oleic acid, palmitoleic acid, linoleic acid, fatty acid mixtures containing these unsaturated fatty acids, etc. These fatty acids can be suitably used in combination. Of these, a liposome containing an unsaturated fatty acid with short side chains is useful for producing a liposome with a small particle size in view of curvature relationship.

The method for causing the liposome of the present invention to encapsulate the trans-resveratrol polysaccharide of the present invention is not particularly limited. The method may be a passive loading technique in which liposomes are formed after the trans-resveratrol polysaccharide of the present invention is mixed with liposome constituent components, or a remote loading technique in which the trans-resveratrol polysaccharide of the present invention and liposomes that have been previously formed are mixed and brought into contact with each other, thereby allowing the liposomes to encapsulate the trans-resveratrol polysaccharide.

The method for forming the above liposome is not particularly limited. Examples include a thin-film hydration method, freeze-dry method, droplet method, AC-electric field-dependent electroformation, ultrasonic method, reverse-phase evaporation method, injection method, spray-dry method, a method using a CO₂/H₂O emulsion, a supercritical water method, a method using a microchannel, etc.

Since the liposome of the present invention encapsulates the above trans-resveratrol polysaccharide, the liposome is expected to directly exhibit the excellent effects of the polysaccharide.

Further, since the liposome of the present invention encapsulates the above trans-resveratrol polysaccharide, compositions comprising the liposome of the present invention as an active ingredient can stably maintain the trans-resveratrol polysaccharide. Therefore, such compositions are expected to exhibit excellent effects based on the effects of the above trans-resveratrol polysaccharide. Furthermore, these compositions are useful in the field of cosmetics or food-and-drink products, as described in detail below regarding the composition of the present invention.

### Composition

The composition of the present invention comprises the trans-resveratrol polysaccharide of the present invention and/or the liposome of the present invention.

The content of the trans-resveratrol polysaccharide or liposome in 100 wt. % of the composition is not particularly limited, as long as the trans-resveratrol polysaccharide exhibits the effects described above. The content can be set within the range of 0.0001 to 100 wt. %. The trans-resveratrol polysaccharide and/or liposome of the present invention can be directly used as the composition of the present invention.

When the composition of the present invention comprises the liposome, the amount of the trans-resveratrol polysaccharide encapsulated in the liposome may be set within the above range. When the composition comprises the trans-resveratrol polysaccharide and the liposome, the total amount of the trans-resveratrol polysaccharide and the trans-resveratrol polysaccharide encapsulated in the liposome may be set within the above range.

The trans-resveratrol polysaccharide of the present invention exhibits effects such as promotion of SOD production, and elimination of singlet oxygen in the body; inhibition of tyrosinase activity, thereby suppressing dopachrome production; and inhibition of hyaluronidase activity, collagenase activity, elastase activity, etc., as described above.

Further, the trans-resveratrol polysaccharide of the present invention exhibits excellent effects such as inhibition of the Maillard reaction, acceleration of the expression of sirtuin gene clusters, and suppression of the expression of NFκB, which is a transcription factor that induces inflammation.

In addition, the trans-resveratrol polysaccharide of the present invention exhibits effects such as inhibition of the growth of acne bacteria, as well as reduction in fat production in the liver and acceleration of fat degradation in the liver.

Moreover, the trans-resveratrol polysaccharide of the present invention is superior to trans-resveratrol aglycones in terms of higher rescue activity from cell death induced by hydrogen peroxide, etc., and less cytotoxicity. The trans-resveratrol polysaccharide of the present invention is also superior to trans-resveratrol aglycones in terms of the whitening effect.

By taking advantage of these effects, the composition of the present invention comprising the trans-resveratrol polysaccharide and/or liposome of the present invention is expected to exhibit the excellent effects of the trans-resveratrol polysaccharide; therefore, the composition of the present invention can be suitably used in the field of cosmetics and food-and-drink products. That is, one embodiment of the composition of the present invention is a cosmetic composition or a food-and-drink composition.

### Cosmetic Composition

The trans-resveratrol polysaccharide and/or liposome of the present invention can be directly used as the cosmetic composition, as described above regarding the composition. Generally, the trans-resveratrol polysaccharide and/or liposome of the present invention, in combination with carriers and additives acceptable in the field of cosmetics, can be prepared in the form of various cosmetics.

The content of the trans-resveratrol polysaccharide in the cosmetic composition of the present invention is generally about 0.01 to a 20 wt. %, and preferably about 0.1 to 10 wt. %.

When the cosmetic composition comprises the liposome, or when the cosmetic composition comprises the trans-resveratrol polysaccharide and the liposome, the amount of the trans-resveratrol polysaccharide encapsulated in the liposome, or the total amount of the trans-resveratrol polysaccharide and the trans-resveratrol polysaccharide encapsulated in the liposome may be set within the range described above regarding the content of the trans-resveratrol polysaccharide in the composition of the present invention.

The form of the cosmetic composition includes various forms of general cosmetics, and is not particularly limited. Examples include lotions (liquids), mousses, gels, jellies, milky lotions, suspensions, creams, ointments, sheets, aerosols, sprays, and the like.

Moreover, the type of the cosmetic composition is not limited, as long as it is an external use composition to be applied to the skin or hair. Examples include makeup cosmetics, such as foundations (emulsification foundations), rouges, and face powders; basic cosmetics, such as face lotions, milky lotions, creams (skin creams), lotions, oils, and pack agents; skin cleansing agents, such as soaps, facial washes, cleansing creams (cleansing gels), and body soaps; hair cosmetics, such as shampoos, hair tonics, rinses, conditioners, styling agents, and hair restorers; massage agents and cleaning agents; detergents; and bath additives, bath agents, and the like.

Since the cosmetic composition of the present invention comprises the trans-resveratrol polysaccharide of the present invention, when applied to the skin (including scalp), hair, etc., the cosmetic composition is expected to exhibit in the body the trans-resveratrol polysaccharide effects described above, such as inhibition of the Maillard reaction; acceleration of the expression of sirtuin gene clusters; suppression of the expression of NFκB, which is a transcription factor that induces inflammation; inhibition of the growth of acne bacteria; and reduction in fat production in the liver and acceleration of fat degradation in the liver.

In addition, the trans-resveratrol polysaccharide of the present invention exhibits a superior whitening effect than resveratrol aglycones.

The cosmetic composition of the present invention is therefore expected to exhibit effects such as skin anti-aging, skin whitening, skin moisturization, retention of skin elasticity, anti-inflammation, suppression of the development or aggravation of pimples, and prevention of metabolic syndrome.

The amount of the cosmetic composition applied can be suitably set on the basis of the sex and age of application subject, the application form of the composition, the degree of expected effect, etc. For example, the application amount may be such that the amount of the trans-resveratrol polysaccharide is generally about 0.01 to 2 mg per cm² of the skin.

Examples of application subjects include persons who desire skin anti-aging, suppression of the development or aggravation of pimples, and prevention of metabolic syndrome, etc.; and further include persons who desire skin whitening, skin moisturization, retention of skin elasticity, anti-inflammation, etc.

### Food-and-Drink Composition

The trans-resveratrol polysaccharide and/or liposome of the present invention can be directly used as the food-and-drink composition, as described above regarding the composition. Generally, the trans-resveratrol polysaccharide and/or liposome of the present invention, in combination with carriers and additives acceptable in the field of food-and-drink products, can be prepared in the form of various food-and-drink products.

The content of the trans-resveratrol polysaccharide in the food-and-drink composition of the present invention can be suitably set as described above regarding the composition of the present invention. The content is generally about 0.01 to 20 wt. %, and preferably about 0.1 to 10 wt. %.

When the food-and-drink composition comprises the liposome, or when the food-and-drink composition comprises the trans-resveratrol polysaccharide and the liposome, the amount of the trans-resveratrol polysaccharide encapsulated in the liposome, or the total amount of the trans-resveratrol polysaccharide and the trans-resveratrol polysaccharide encapsulated in the liposome may be set within the range described above regarding the content of the trans-resveratrol polysaccharide in the composition of the present invention.

Examples of the food-and-drink composition of the present invention include general food-and-drink products, food for specified health use (including conditional food for specified health use), supplements, functional food, food for patients (including supplements), etc.

Specific examples of the food-and-drink composition include drinks, such as soft drinks, carbonated drinks, energy drinks, fruit drinks, and lactic-acid drinks; frozen desserts, such as ice cream and shaved ice; confectionery, such as gum, chocolate, candies, tablet candies, snacks, jellies, jams, creams, and gummy candies; noodles, such as soba, udon, and instant noodles; fishery and livestock processed foods, such as kamaboko, ham, and sausage; dairy products, such as processed milk and fermented milk; fats and oils, and fat-and-oil processed foods, such as salad oil, mayonnaise, whipped cream, and dressing; seasonings, such as marinade sauce and basting sauce; and soup, salad, side dishes, pickles, bread, cereal, and the like.

Furthermore, for example, in the case of food for specified health use, supplements, functional food, etc., the food-and-drink composition of the present invention may be in the form of preparations, such as solid preparations (e.g., powders, granules, capsules, troches, and tablets) and liquid preparations (e.g., syrups and drinks).

Since the food-and-drink composition of the present invention comprises the trans-resveratrol polysaccharide of the present invention, when eaten, the food-and-drink composition is expected to exhibit in the body the effects described above, such as inhibition of the Maillard reaction; acceleration of the expression of sirtuin gene clusters; suppression of the expression of NFκB, which is a transcription factor that induces inflammation; inhibition of the growth of acne bacteria; and reduction in fat production in the liver and acceleration of fat degradation in the liver.

In addition, the trans-resveratrol polysaccharide of the present invention exhibits a superior whitening effect than resveratrol aglycones.

The food-and-drink composition of the present invention is therefore expected to exhibit effects such as skin anti-aging, skin whitening, skin moisturization, retention of skin elasticity, anti-inflammation, suppression of the development or aggravation of pimples, and prevention of metabolic syndrome.

The dosage of the food-and-drink composition is generally about 5 to 20 mg, and preferably about 10 to 20 mg, calculated in terms of the amount of the trans-resveratrol polysaccharide per dose. Moreover, the food-and-drink composition may be taken once or several times (e.g., two or three times) per day. The amount of the trans-resveratrol polysaccharide mixed into the food-and-drink composition can be adjusted so as to satisfy the above dosage range.

Subjects to which the food-and-drink composition of the present invention is applied are not particularly limited; however, examples include persons who desire skin anti-aging, suppression of the development or aggravation of pimples, and prevention of metabolic syndrome, etc.; and further include persons who desire skin whitening, skin moisturization, retention of skin elasticity, anti-inflammation, etc.

### EXAMPLES

Examples are provided below to describe the present invention in more detail. The present invention, however, is of course not limited to the following Examples.

### Production Example 1

400 mg of trans-resveratrol-3-O-β-D-monoglucoside, 4 g of α-cyclodextrin, and 25 mL of Cyclodextrin glucanotransferase “Amano” (600 units/mL; Amano Enzyme Inc.) were added to 300 mL of citric acid-sodium citrate buffer (pH of 5.4), and the mixture was stirred by a magnetic stirrer in a hot-water bath at 55° C. for 24 hours to perform enzyme reaction.

Subsequently, the reaction solution was heated to 80° C. in a hot-water bath to inactivate the Cyclodextrin glucanotransferase “Amano”. The reaction solution was then cooled to ordinary temperature, and partition extraction with ethyl acetate/water was performed six times. The obtained oil phase fraction was subjected to salting-out, dehydration, and a vacuum concentration process, and sampled by HPLC using a CrestPak C18S column (JASCO; 4.6×150).

The aqueous phase fraction obtained by partition extraction with ethyl acetate was further subjected to partition extraction with water-saturated n-butanol three times, and then to a vacuum concentration process. Subsequently, fractionation was performed by HPLC using a CrestPak C18S column (JASCO; 4.6×150), and each fraction was sampled. The HPLC was performed for 40 minutes under the following conditions: column temperature: 40° C., mobile phase: a mixture of acetonitrile and water (15:85), flow rate: 1.0 m/min, and injection amount: 5 μL.

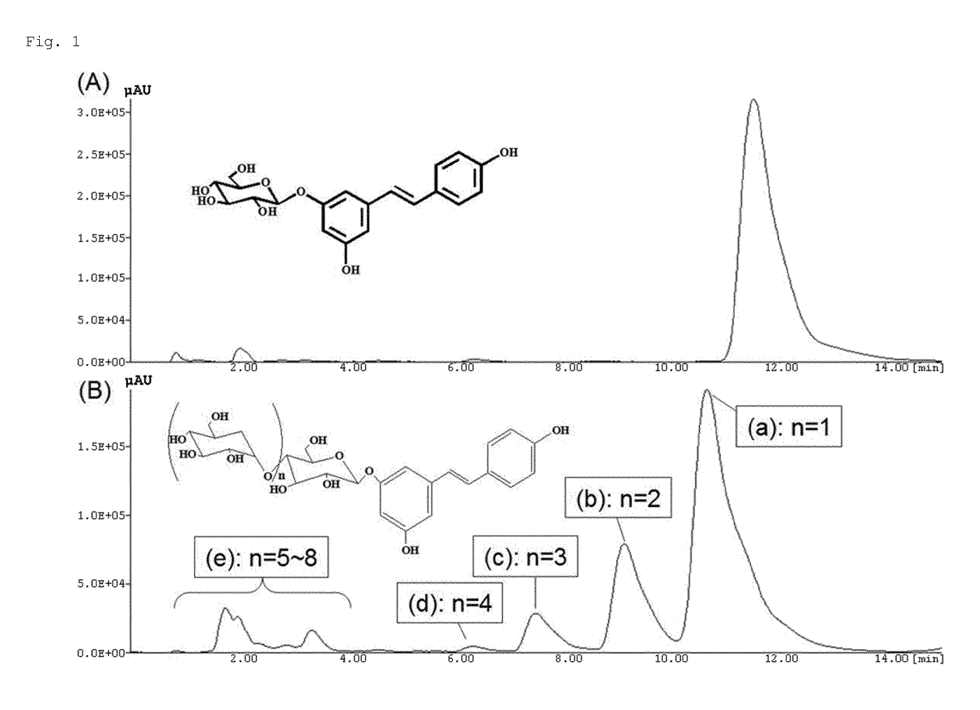

FIG. 1 shows the results of column chromatography by HPLC of the reactant obtained by using trans-resveratrol-3-O-β-D-monoglucoside as a starting material. FIG. 1 (A) is the column chromatogram of the oil phase fraction after phase separation with ethyl acetate/water, and (B) is the column chromatogram of the aqueous phase fraction.

As shown in FIG. 1 (A), only a single peak was obtained from the oil phase fraction, whereas (B) shows that prominent peaks, which indicated that sugar units were added to the trans-resveratrol-3-monoglucoside to produce polysaccharides with 2 to 5 sugars, were detected on the basis of the difference in retention time by HPLC (fractions indicated by (a) to (d)). Further, the production of polysaccharides with 6 to 9 sugars was also detected (fraction (e)).

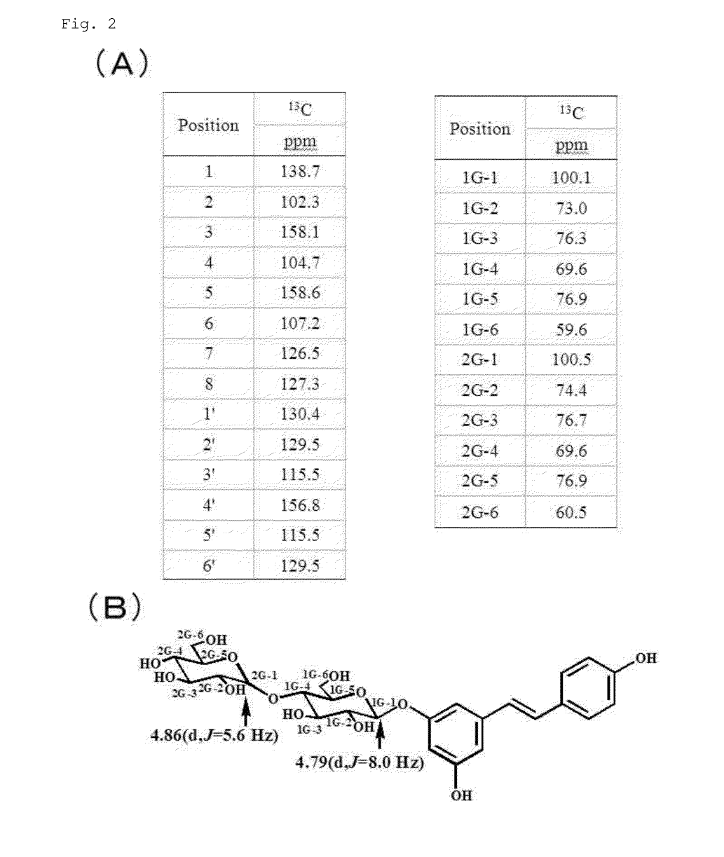

The fraction (a), which showed the longest retention time (horizontal axis of the graph) in the column chromatogram by HPLC of the oil phase fraction, was subjected to NMR measurement. The frequency of the apparatus used was 400 MHz. FIG. 2 shows the results.

From the results shown in FIG. 2 (A), this fraction was identified as trans-resveratrol-3-O-β-D-diglucoside shown in (B). These results also suggested that the fraction showing a single peak in FIG. 1 (A) was trans-resveratrol-3-O-β-D-monoglucoside, which was a starting material.

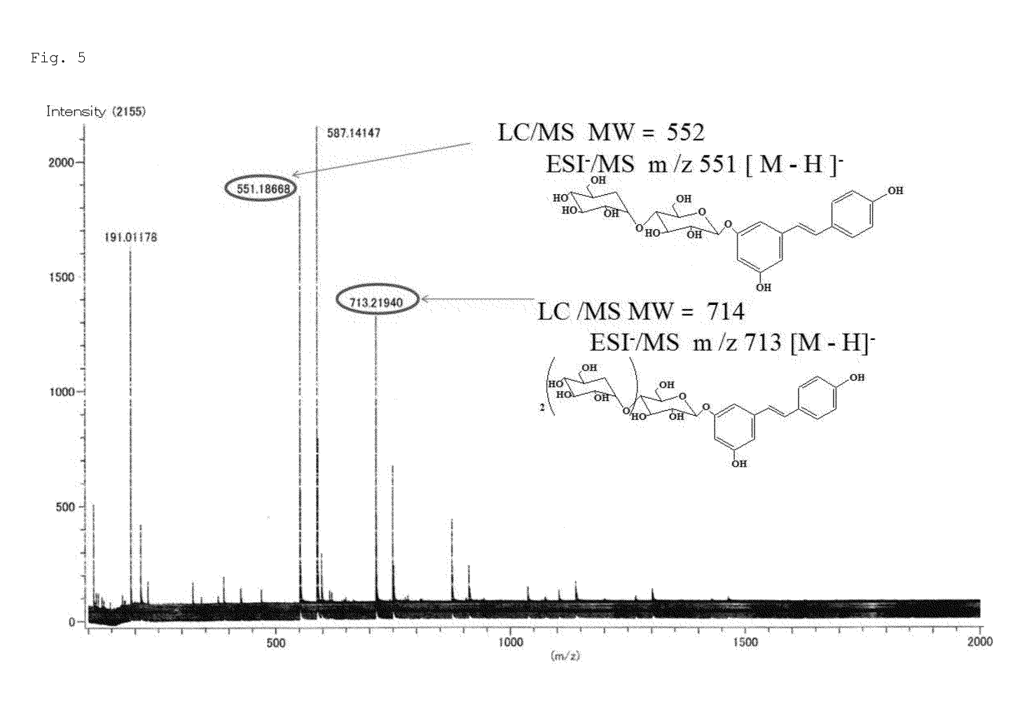

Further, the fraction (a) and the fraction (b), which showed the second-longest retention time, were combined and subjected to MS measurement. FIG. 5 shows the results. The obtained results matched the molecular weights of trans-resveratrol-3-O-β-D-diglucoside and a polysaccharide in which two molecules of glucose were added to trans-resveratrol-3-O-β-D-monoglucoside.

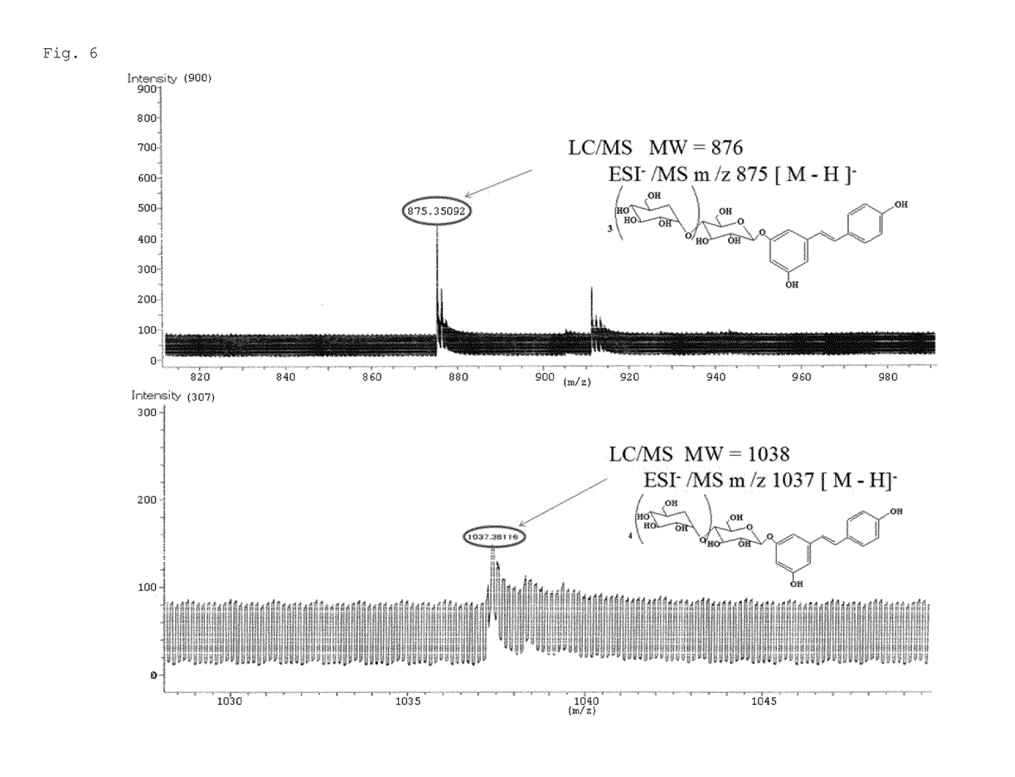

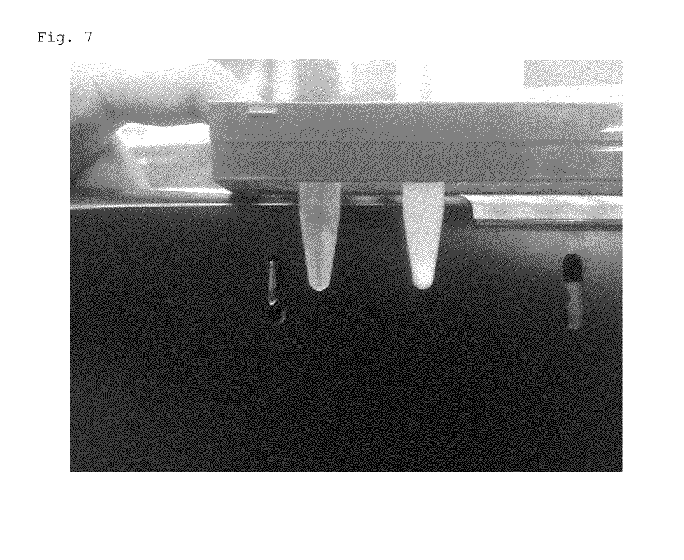

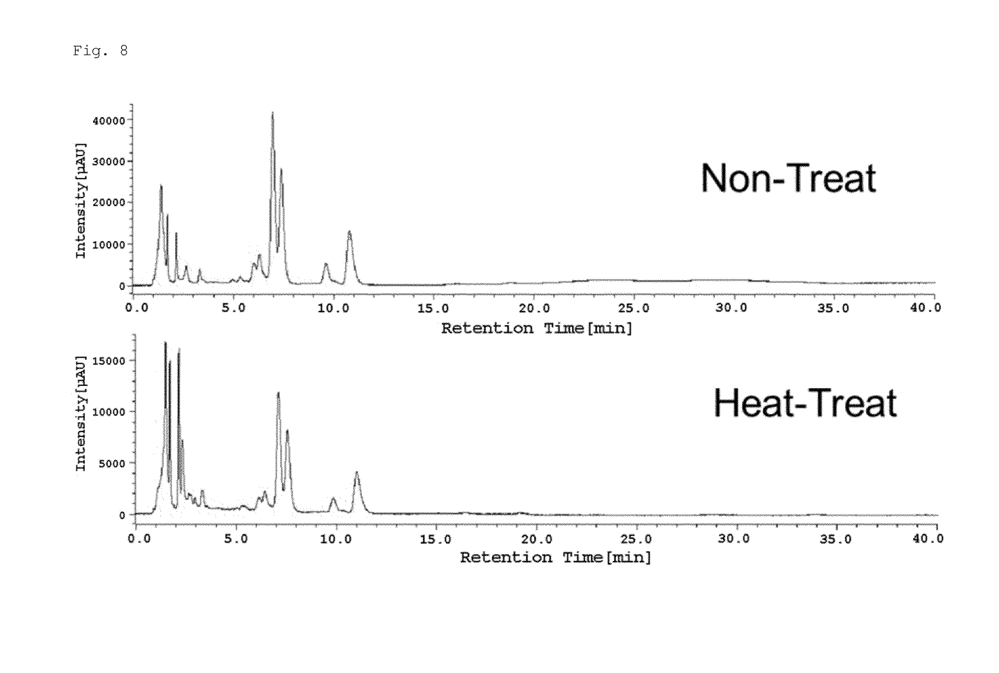

FIG. 6 shows the results when the fractions (c) and (d) were similarly subjected to MS measurement. The obtained results of these fractions matched the molecular weights of a polysaccharide in which three molecules of glucose were added to trans-resveratrol-3-O-β-D-monoglucoside, and a polysaccharide in which four molecules of glucose were added to trans-resveratrol-3-O-β-D-monoglucoside.

The above results revealed that although trans-resveratrol monoglucoside was not dissolved in water at ordinary temperature, polysaccharides in which one or more glucose molecules were further added to trans-resveratrol monoglucoside (e.g., trans-resveratrol-3-O-β-D-diglucoside), etc., could acquire water solubility at ordinary temperature.

FIG. 7 is a photographic image showing trans-resveratrol-3-O-β-D-monoglucoside contained in the fraction showing a single peak detected in FIG. 1 (A), and trans-resveratrol-3-O-β-D-diglucoside contained in the fraction (a) in FIG. 1 (B), after they were dissolved in distilled water and allowed to stand at room temperature.

As is clear from the figure, the tube of the trans-resveratrol-3-O-β-D-monoglucoside was turbid (on the right side of the photographic image), indicating that it was not dissolved in distilled water at room temperature, whereas the tube of the trans-resveratrol-3-O-β-D-diglucoside was transparent (on the left side of the photographic image), indicating that it was easily dissolved in distilled water at room temperature.

The retention time of cis-resveratrol by column chromatography is known to tend to be longer than that of trans-resveratrol; however, the results of the above chromatogram showed no detection of cis-resveratrol.

### Production Example 2

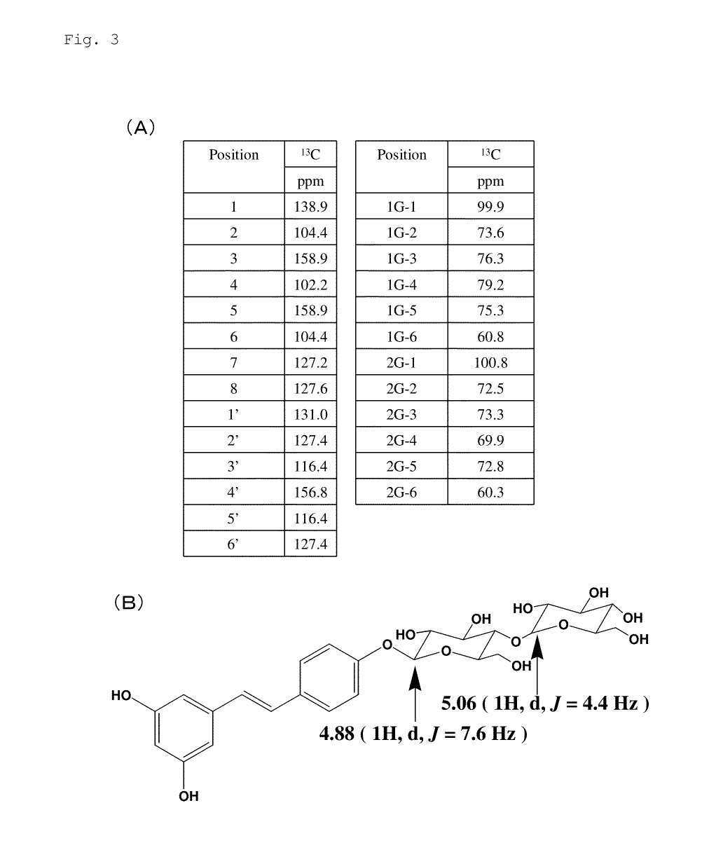

Moreover, the starting material in Production Example 1 was changed from trans-resveratrol-3-monoglucoside to trans-resveratrol-4′-monoglucoside, and the same experiment was performed. FIG. 3 shows the results of the NMR measurement of a fraction with a peak indicating a polysaccharide with 2 sugars, fractionated by HPLC.

From the results shown in FIG. 3 (A), trans-resveratrol-4′-O-β-D-diglucoside shown in FIG. 3 (B) was identified.

In a column chromatography experiment the same as in Production Example 1, cis-resveratrol was also not detected.

### Comparative Example

70 g of soybean cells (E. perriniana) and 1 mol of trans-resveratrol (Wako Pure Chemical Industries, Ltd., Japan) were placed in a 300-mL flask, and suspension culture was performed at 25° C. for 5 days.

The culture medium and cultured cells were collected every 12 hours after culture. The culture medium was subjected to an extraction process using ethyl acetate. The cultured cells were lysed in methanol, and partition extraction was performed using water and ethyl acetate.

Both ethyl acetate fractions were combined and subjected to HPLC using a Diaion-HP20 column (Mitsubishi Chemical Corporation), and the amounts of trans-resveratrol, which was used as a starting material, and derivatives thereof were calculated from each fraction, together with NMR data.

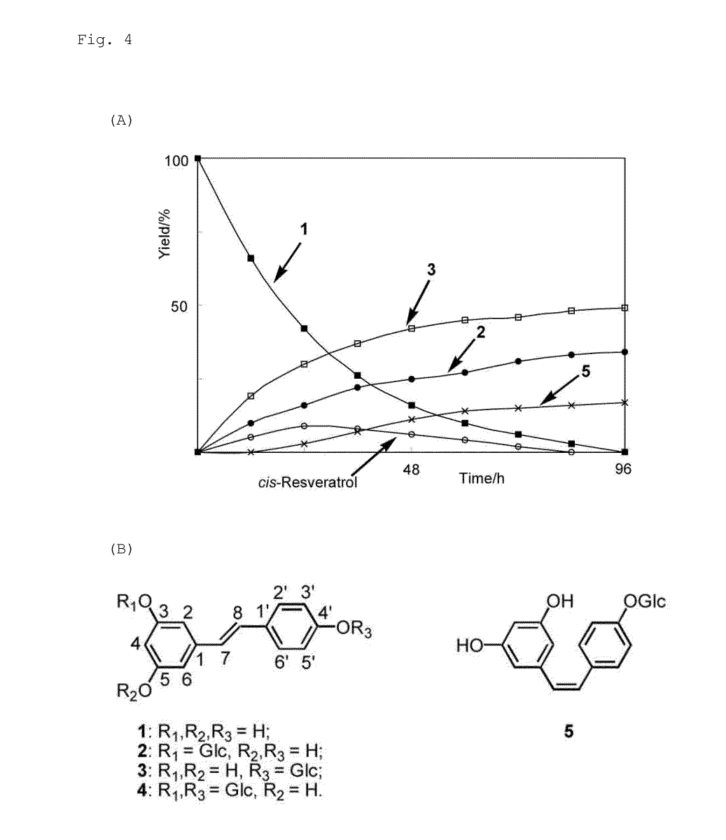

FIG. 4 shows the results. NMR-assignment data of the above trans-resveratrol derivatives shown in FIG. 4 are as follows:

### trans-Resveratrol-3-O-β-D-glucopyranoside (2)

¹H NMR (400 MHz, DMSO-d6): δ3.15-3.53 (5H, m, H-2″,3″,4″,5″,6a″), 3.74 (1H, dd, J=12.0, 2.0 Hz, H-6b″), 4.80 (1H, d, J=7.6 Hz, H-1″), 6.33 (1H, m, H-4), 6.56 (1H, d, J=1.8 Hz, H-6), 6.73 (1H, d, J=1.8 Hz, H-2), 6.76 (2H, d, J=8.8 Hz, H-3′,5′), 6.86 (1H, J=16.8 Hz, H-7), 7.03 (1H, d, J=16.0 Hz, H-8), 7.39 (2H, d, J=8.8 Hz, H-2′,6′).

¹³CNMR (100 MHz, DMSO-d6): δ60.8 (C-6″), 69.8 (C-4″), 73.3 (C-2″), 76.7 (C-3″), 77.1 (C-5″), 100.6 (C-1″), 102.7 (C-2), 104.7 (C-4), 107.1 (C-6), 115.5 (C-3′, C-5′), 125.1 (C-7), 127.9 (C-2′, C-6′), 128.5 (C-8), 129.9 (C-1′), 139.3 (C-1), 157.2 (C-4′), 158.2, 158.8 (C-3, C-5).

HRFABMS: m/z 413.1225 [M+Na]⁺.

### trans-Resveratrol-4′-O-β-D-glucopyranoside (3)

¹H NMR (DMSO-d6): δ3.22-3.50 (5H, m, H-2″,3″,4″,5″,6a″), 3.70 (1H, dd, J=11.6, 1.8 Hz, H-6b″), 4.88 (1H, d, J=7.6 Hz, H-1″), 6.01 (1H, m, H-4), 6.41 (2H, d, J=1.8 Hz, H-2,6), 6.95 (2H, d, J=14.8 Hz, H-7,8), 7.01 (2H, d, J=8.8 Hz, H-3′,5′), 7.50 (2H, d, J=8.8 Hz, H-2′,6′).

¹³C NMR (DMSO-d6): δ60.7 (C-6″), 69.7 (C-4″), 73.2 (C-2″), 76.6 (C-3″), 77.0 (C-5″), 100.2 (C-1″), 102.0 (C-4), 104.4 (C-2, C-6), 116.3 (C-3′, C-5′), 127.1, 127.3 (C-7, C-8), 127.5 (C-2′, C-6′), 130.7 (C-1′), 138.9 (C-1), 156.8 (C-4′), 158.4 (C-3, C-5).

HRFABMS: m/z 413.1219 [M+Na]⁺.

### cis-Resveratrol-4′-O-β-D-glucopyranoside (5)

¹H NMR (400 MHz, CD3OD): δ3.14-3.87 (6H, m, H-2″,3″,4″,5″,6″), 4.90 (1H, d, J=7.2 Hz, H-1″), 6.22 (1H, t, J=2.0 Hz, H-4), 6.30 (2H, d, J=2.4 Hz, H-2,6), 6.38 (1H, d, J=12 Hz, H-7), 6.49 (1H, d, J=12 Hz, H-8), 6.99 (2H, d, J=8.0 Hz, H-3′,5′), 7.20 (2H, d, J=8.0 Hz, H-2′,6′);

¹³C NMR (100 MHz, CD3OD): 662.5 (C-6″), 70.9 C-4″), 75.0 (C-2″), 77.8 (C-3″), 78.2 (C-5″), 101.1 (C-1″), 102.5 (C-4), 108.5 (C-2, C-6), 117.5 (C-3′, C-5′), 127.9 (C-7), 130.5 (C-8), 131.3 (C-2′, C-6′), 133.2 (C-1′), 141.0 (C-1), 158.0 (C-4′), 159.5 (C-3, C-5).

HRFABMS: m/z 413.1215 [M+Na]⁺.

The results shown in FIG. 4 (A) revealed that within 24 hours after culture, the amount of trans-resveratrol (1) as a starting material decreased, trans-resveratrol-3-O-β-D-glucoside (2) and trans-resveratrol-4′-O-β-D-glucoside (3) were generated, further cis-resveratrol-4′-O-β-D-glucoside (5) was generated, and the final yield of cis-resveratrol was 17%.

Moreover, from the results shown in FIG. 4 (A), cis-resveratrol was also identified. The above results revealed that in the case of resveratrol aglycone and resveratrol monosaccharide glycoside, the trans-form and the cis-form coexisted.

### Example 1

The aqueous phase fraction after phase separation with ethyl acetate/water obtained in Production Example 1 (see FIG. 1 (B)) was subjected to heat treatment at 80° C. overnight. The samples before and after treatment were subjected to column chromatography in the same manner as described above. FIG. 8 shows the results.

When the column chromatography results were compared before and after heat treatment, no change was observed in the chart pattern. In consideration of the tendency of cis-resveratrol to show a longer retention time, as described above, it was revealed that the above trans-resveratrol polysaccharides (glycosides with 2 or more sugars) resisted isomerization to the cis-form by heat treatment, and were stable as the trans-form.

This suggests that trans-resveratrol polysaccharides are not isomerized to the cis-form by the action of ultraviolet radiation, acid, base, etc., other than heat treatment. Trans-resveratrol polysaccharides that can withstand such action are useful because they are stable as the trans-form that exhibits antioxidant action when mixed into cosmetics, food, etc.

### Example 2

An experiment was conducted to confirm the safety of trans-resveratrol polysaccharide. Trans-resveratrol (“RSV”), trans-resveratrol-O-β-D-3-monoglucoside-4′-diglycoside (“4M-RSV”), and a polysaccharide mixture in which two or more glucose molecules were linked to the 3 position of trans-resveratrol (“3P-RSV”) were each added to a culture medium of 3T3 mouse fibroblasts so that the final concentration was 200 μM, and the fibroblasts were cultured with 5% carbon dioxide at 37° C. for 12 hours.

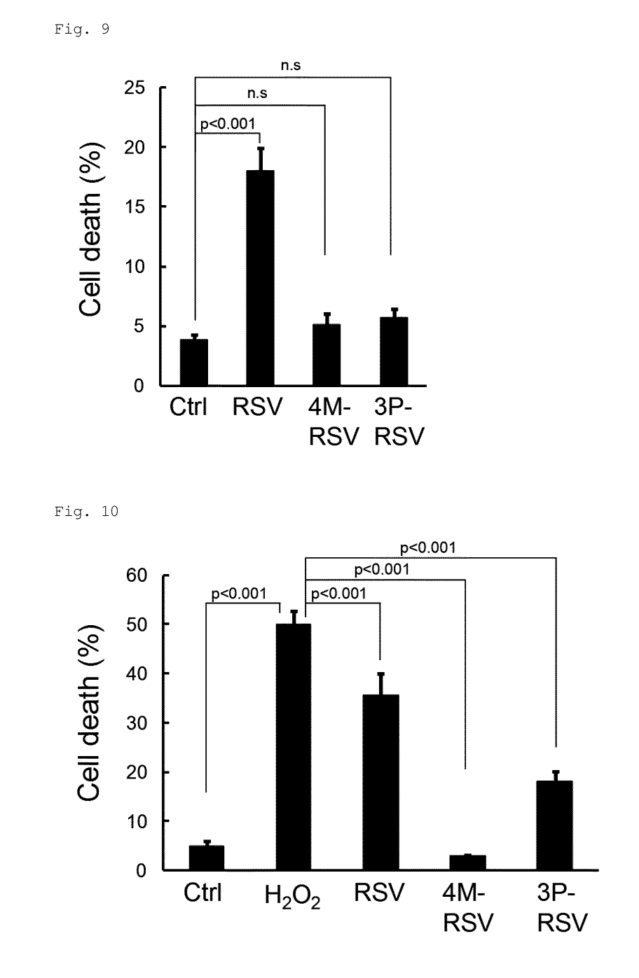

Subsequently, the cultured cells were fixed with 4% paraformaldehyde and stained with Hoechst. Cells in which aggregation in the cell nucleus was observed were determined to be cell death, and the percentage of cell death (vertical axis in the graph) was calculated. FIG. 9 shows the results.

The graph shown in FIG. 9 revealed that trans-resveratrol polysaccharide had significantly less cytotoxicity than trans-resveratrol (aglycone).

### Example 3

An experiment was conducted to confirm the trans-resveratrol polysaccharide's rescue activity from cell death. RSV, 4M-RSV, and 3P-RSV were each added to a culture medium of 3T3 mouse fibroblasts so that the final concentration was 10 μM, and the fibroblasts were cultured with 5% carbon dioxide at 37° C. for 3 hours. Subsequently, hydrogen peroxide was added to the culture medium so that the final concentration was 15 μM, thereby inducing cell death.

After culture for 12 hours, cell death was determined in the same manner as in Example 1, and the percentage of cell death was calculated. FIG. 10 shows the results.

The graph shown in FIG. 10 revealed that trans-resveratrol polysaccharide had higher rescue activity from cell death induced by hydrogen peroxide than trans-resveratrol (aglycone). In particular, 4M-RSV only showed cell death almost equal to that of the case in which hydrogen peroxide was not added (Ctrl); therefore, it was revealed that 4M-RSV exhibited particularly excellent rescue activity.

### Example 4

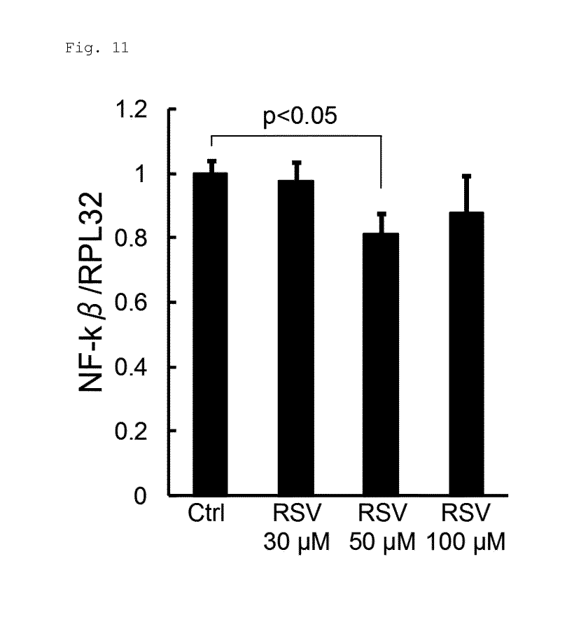

An experiment was conducted to confirm the effect of suppressing the expression of NFκB, which is a transcription factor that induces inflammation. Epithelial tissue models were produced using a normal human three-dimensional epithelial tissue model production kit MelanoDerm (MEL-300; Kurabo). RSV was added to the culture media so that the final concentration was 0 μM (Ctrl), 30 μM, 50 μM, and 100 μM, and the tissue models were cultured according to the manual of the kit. RNA in the skin tissue models was collected three weeks after the addition of RSV, and subjected to quantitative PCR analysis to measure the expression level of NFκB. FIG. 11 shows the results.

The graph shown in FIG. 11 revealed that RSV suppressed the expression level of NFκB in the epithelial tissue models in a concentration-dependent manner.

### Example 5

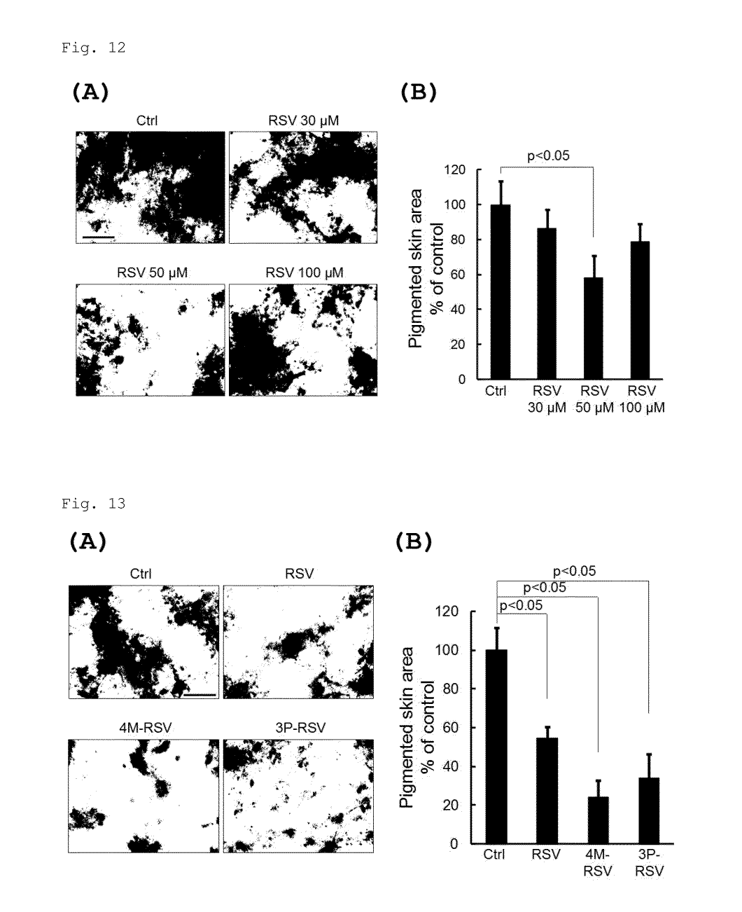

Epithelial tissue models were produced using the above kit. RSV was added to the culture media so that the final concentration was 0 μM (Ctrl), 30 μM, 50 μM, and 100 μM, and the tissue models were cultured according to the manual of the kit. Photographic images of the epithelial tissue models were taken three weeks after the addition of RSV. The degree of reduction in black areas due to the addition of RSV was calculated using ImageJ image-analysis software, and the results were graphed. FIG. 12 shows the results.

The results shown in FIG. 12 revealed that RSV reduced black areas in the epithelial tissue models in a concentration-dependent manner. It was thus revealed that RSV had a whitening effect.

### Example 6

Epithelial tissue models were produced in the same manner as in Example 5. RSV, 4 M-RSV, and 3P-RSV were each added so that the final concentration was 50 μM, and the skin tissue models were observed. FIG. 13 shows photographic images and the calculated degree of reduction in black areas. The graph of FIG. 13 shows the results of conversion of RSV, 4 M-RSV, and 3P-RSV to the degree of reduction per molecular unit of RSV.

The results shown in FIG. 13 revealed that trans-resveratrol polysaccharide exhibited a superior whitening effect than trans-resveratrol (aglycon). In particular, it was revealed that 4M-RSV exhibited a significantly excellent whitening effect.

### Formulation Examples

In the following, face lotions, creams, soaps, cleansing gels, pack agents, emulsification foundations, hair tonics, and bath agents (all of which are cosmetic compositions) comprising the trans-resveratrol polysaccharide of the present invention (noted as “resveratrol polysaccharide” in the following tables), including the trans-resveratrol-3-O-β-D-diglucoside, trans-resveratrol-4′-O-β-D-diglucoside, etc., prepared in the above Examples, are produced according to the formulation examples shown in Tables 1 to 9 by using known methods; and tablets, gummy candies, jellies, candies, and soft drinks (all of which are food-and-drink compositions) are produced according to the formulation examples shown in Tables 10 to 13 by using known methods. Further, liposomes encapsulating the trans-resveratrol polysaccharide are produced according to the formulation example shown in Table 14.

### Formulation Example 1

Components A shown below are mixed and dissolved to prepare Solution A. Separately, Components B shown below are mixed and dissolved to prepare Solution B. Solutions A and B are uniformly mixed to prepare a total of 100 parts by mass of face lotions.

TABLE 1
Face lotion

| | | |
|---|---|---|
| Component A | Polyoxyethylene sorbitan laurate | 1.2 |
| | Ethyl alcohol | 4.0 |
| Component B | Resveratrol polysaccharide | 1.5 |
| | Glycerin | 4.0 |
| | Phenoxyethanol | 0.2 |
| | 1,3-butylene glycol | 4.0 |
| | Purified water | Remnant |

### Formulation Example 2

Components A and B shown below are separately heated and dissolved to prepare Solutions A and B, respectively. Solution B is added to Solution A, and the mixture is emulsified, and cooled while stirring, thereby preparing a total of 100 parts by mass of creams.

TABLE 2
Cream

| | | |
|---|---|---|
| Component A | Squalane | 4.0 |
| | Methyopolysiloxane | 0.5 |
| | (caprylic-capric acid)glyceryl | 12.0 |
| | Monostearate glyceryl | 4.0 |
| | Monostearate diglyceryl | 2.5 |
| | Monostearate decaglyceryl | 3.0 |
| Component B | Resveratrol polysaccharide | 3.0 |
| | Glycerin | 2.0 |
| | Phenoxyethanol | 0.2 |
| | 1,3-butylene glycol | 3.0 |
| | Purified water | Remnant |

### Formulation Example 3

The following components are mixed by using a known method for soap production, thereby preparing a total of 100 parts by mass of soaps.

TABLE 3

| | | |
|---|---|---|
| Component | Resveratrol polysaccharide | 2.5 |
| | Soap base | 55.0 |
| | Sucrose | 18.5 |
| | Concentrated glycerin | 6.5 |
| | Hydroxyethane diphosphonic acid | 0.2 |
| | Purified water | Remnant |

### Formulation Example 4

Components A and B shown below are separately heated and dissolved to prepare Solutions A and B, respectively. Solution B is added to Solution A, and the mixture is stirred until it becomes homogeneous. The mixture is cooled while stirring, thereby preparing a total of 100 parts by mass of cleansing gels.

TABLE 4

| | | |
|---|---|---|
| Component A | Monomyristate hexaglyceryl | 19.0 |
| | Liquid paraffin | 59.0 |
| | Paraoxybenzoic acid ester | 0.3 |
| Component B | Resveratrol polysaccharide | 0.5 |
| | Concentrated glycerin | 6.0 |
| | Sorbitol | 4.0 |
| | Purified water | Remnant |

### Formulation Example 5

Phase A, Phase B, and Phase C are each homogeneously dissolved. Phase B is added to Phase A for solubilization, and Phase C is then added and homogeneously dissolved, thereby preparing a total of 100 parts by mass of pack agents.

TABLE 5

| | | |
|---|---|---|
| Phase A | Dipropylene glycol | 5.0 |
| | Polyoxyethylene-hardened castor oil | 3.0 |
| Phase B | Olive oil | 4.0 |
| | Tocopherol acetate | 0.1 |
| | Paraoxybenzoic acid ester | 0.2 |
| Phase C | Resveratrol polysaccharide | 4.0 |
| | Sodium hydrogensulfite | 0.03 |
| | Polyvinyl alcohol | 11.0 |
| | Ethanol | 5.0 |
| | Purified water | Remnant |

### Formulation Example 6

Components A shown below are sufficiently mixed and milled to prepare Powder A. Solution B is prepared from Components B, and Solution C is prepared from Components C. After Solution C is heated and stirred, Powder A is added thereto, followed by homomixer treatment. Further, heated and mixed Solution B is added thereto, followed by homomixer treatment. The resultant is cooled while stirring to room temperature, thereby preparing a total of 100 parts by mass of emulsification foundations.

TABLE 6

| | | |
|---|---|---|
| Component A | Titanium dioxide | 10.5 |
| | Sericite | 5.5 |
| | Kaolin | 3.0 |
| | Yellow iron oxide | 0.6 |
| | Red oxide | 0.4 |
| | Black iron oxide | 0.2 |
| Component B | Decamethylcyclopentasiloxane | 11.5 |
| | Liquid paraffin | 7.0 |
| Component C | Resveratrol polysaccharide | 1.0 |
| | Sorbitan sesquioleate | 3.0 |
| | 1,3-butylene glycol | 6.0 |
| | Paraoxybenzoic acid ester | 0.2 |
| | Purified water | Remnant |

### Formulation Example 7

Components B shown below are added to Component A, and the mixture is stirred and dissolved. Components C are then added and further stirred, thereby preparing a total of 100 parts by mass of hair tonics.

TABLE 7

| | | |
|---|---|---|
| Component A | Ethanol | 40.0 |
| Component B | Resveratrol polysaccharide | 2.5 |
| | Glycerin | 3.0 |
| | L-menthol | 0.1 |
| Component C | Purified water | Remnant |

### Formulation Example 8

A total of 100 parts by mass of bath agents is prepared using the following components by using a known method.

TABLE 8

| | | |
|---|---|---|
| Component | Resveratrol polysaccharide | 1.0 |
| | Sodium hydrogencarbonate | Remnant |
| | Dried sodium sulfate | 45.0 |
| | Light anhydrous silicic acid | 0.4 |
| | Flavoring agent | 1.3 |

### Formulation Example 9

Components A shown below are each sieved and mixed, and Component B is then added and mixed. Subsequently, the mixture is tabletted by using a known method, thereby preparing a total of 600 mg of tablets.

TABLE 9

| | | |
|---|---|---|
| Component A | Resveratrol polysaccharide | 200 |
| | Reduced maltose starch syrup | 340 |
| | Lactose | 20 |
| | Corn starch | 25 |
| Component B | Glycerin fatty acid ester | 15 |

### Formulation Example 10

A total of 750 mL of jellies is prepared by using a known method according to the following formulation.

TABLE 10

| Component | Resveratrol polysaccharide | 1.0 | g |
|---|---|---|---|
| | Gelling agent | 3.5 | g |
| | Sugar | 50 | g |
| | Fruit juice | 10 | g |
| | Flavoring agent, coloring agent | Suitable | |
| | | amount | |
| | Acidulant, sweetener | Suitable | |
| | | amount | |
| | Purified water | 1,000 | mL |
| | Produced amount | 750 | mL |

### Formulation Example 11

A total of 100 parts by weight of gummy candies is prepared by using a known method according to the following formulation.

TABLE 11

| | | |
|---|---|---|
| Component | Resveratrol polysaccharide | 1.0 |
| | Reduced syrup | 35.0 |
| | Granulated sugar | 20.0 |
| | Glucose | 20.0 |
| | Gelatin | 4.7 |
| | Fruit juice | 4.0 |
| | Flavoring agent, coloring agent | Suitable |
| | amount | |
| | Purified water | Remnant |

### Formulation Example 12

A total of 100 parts by mass of candies is prepared by using a known method according to the following formulation.

TABLE 12

| | | |
|---|---|---|
| Component | Resveratrol polysaccharide | 1.0 |
| | Syrup | 35.0 |
| | Sugar | 20.0 |
| | Organic acid | 2.0 |
| | Flavoring agent, coloring agent | Suitable |
| | | amount |
| | Purified water | Remnant |

### Formulation Example 13

A total of 50 mL of soft drinks is prepared by using a known method according to the following formulation.

TABLE 13

| Component | Resveratrol polysaccharide | 10 | mg |
|---|---|---|---|
| | Collagen | 6,000 | mg |
| | High-fructose corn syrup | Suitable | |
| | | amount | |
| | Flavoring agent, coloring agent | Suitable | |
| | | amount | |
| | Purified water | Making the | |
| | | total amount | |
| | | 50 | mL |

### Formulation Example 14

A total of 100 parts by mass of liposomes is prepared by using a known method according to the following formulation.

TABLE 14

| | | |
|---|---|---|
| Component | Resveratrol polysaccharide | 1.0 |
| | Hydrogenated soybean phospholipid | 3.0 |
| | Cholesterol | 0.5 |
| | Purified water | Remnant |

1. At least one trans-resveratrol polysaccharide selected from the group consisting of:
trans-resveratrol-3-O-β-D-diglucoside, and compounds in which sugar is further linked to the hydroxyl group at the 2G-4 position of trans-resveratrol-3-O-β-D-diglucoside via glycoside linkage;
trans-resveratrol-4′-O-β-D-diglucoside, and compounds in which sugar is further linked to the hydroxyl group at the 2G-4 position of trans-resveratrol-4′-O-β-D-diglucoside via glycoside linkage;
trans-resveratrol-O-β-D-3-diglucoside-4′-monoglycoside, and compounds in which sugar is further linked to the hydroxyl group at the 2G-4 position of trans-resveratrol-O-β-D-3-diglucoside-4′-monoglycoside via glycoside linkage;
trans-resveratrol-O-β-D-3-monoglucoside-4′-diglycoside, and compounds in which sugar is further linked to the hydroxyl group at the 2G′-4 position of trans-resveratrol-O-β-D-3-monoglucoside-4′-diglycoside via glycoside linkage; and
trans-resveratrol-O-β-D-3-diglucoside-4′-diglycoside, and compounds in which sugar is further linked to the hydroxyl group at the 2G-4 position and/or the 2G′-4 position of trans-resveratrol-O-β-D-3-diglucoside-4′-diglycoside via glycoside linkage.

2. The trans-resveratrol polysaccharide according to claim 1, wherein the sugar is at least one monosaccharide selected from the group consisting of glucose, galactose, mannose, xylose, fructose, rhamnose, arabinose, allose, altrose, idose, N-acetylglucosamine, N-acetylgalactosamine, talose, glucuronic acid, glucosamine, galactosamine, and fucose; or a sugar in which two or more of the same or different members of these monosaccharides are linked together.

3. The trans-resveratrol polysaccharide according to claim 1, wherein the trans-resveratrol polysaccharide has a sugar chain of 2 to 10 linked monosaccharides.

4. The trans-resveratrol polysaccharide according to claim 1, which is represented by formulae (1) to (3) below: 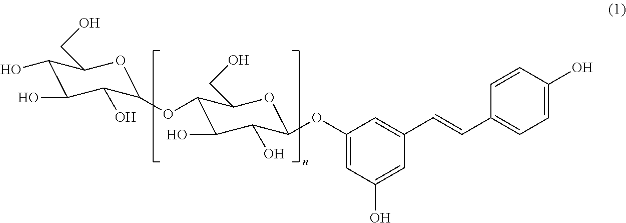
wherein n is an integer of 1 to 9; 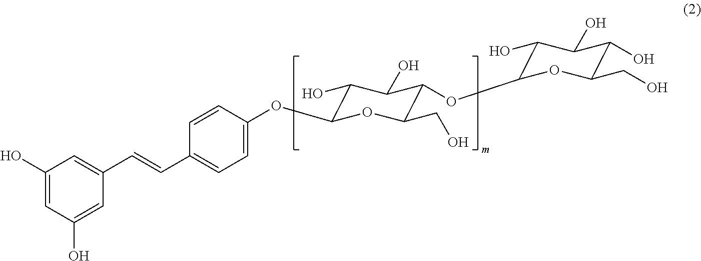
wherein m is an integer of 1 to 9; and 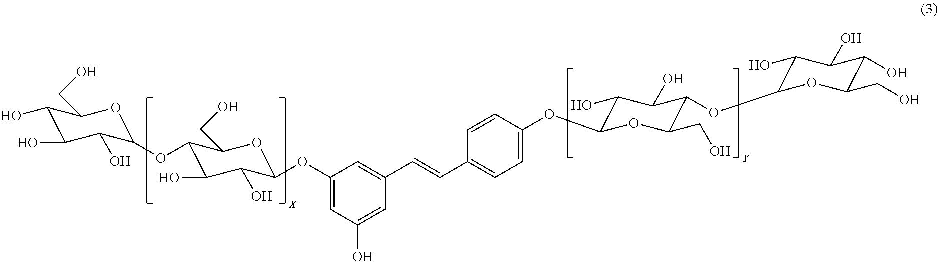
wherein X and Y are each an integer of 1 to 9; and when X is 0, Y is an integer of 1 to 9, and when Y is 0, X is an integer of 1 to 9.

5. A liposome encapsulating the trans-resveratrol polysaccharide according to claim 1.

6. A composition comprising the trans-resveratrol polysaccharide according to claim 1.

7. The composition according to claim 6, which is a food-and-drink composition or a cosmetic composition.

8. A method for producing the trans-resveratrol polysaccharide according to claim 1, the method comprising the step of bringing a trans-resveratrol glucoside into contact with sugar in the presence of γ-cyclodextrin glucanotransferase.

9. The production method according to claim 8, wherein the trans-resveratrol glucoside is at least one selected from the group consisting of trans-resveratrol-3-O-β-D-monoglucoside, trans-resveratrol-4′-O-β-D-monoglucoside, and trans-resveratrol-3,4′-O-β-D-bis-diglucoside.

10. The production method according to claim 8, wherein the sugar is at least one monosaccharide selected from the group consisting of glucose, galactose, mannose, xylose, fructose, rhamnose, arabinose, allose, altrose, idose, N-acetylglucosamine, N-acetylgalactosamine, talose, glucuronic acid, glucosamine, galactosamine, and fucose; or a sugar in which two or more of the same or different members of these monosaccharides are linked together.

11. The production method according to claim 10, wherein the sugar is a sugar in which two or more monosaccharides are linked together, and the sugar has a terminal glucose residue.

12. The production method according to claim 8, wherein the sugar is at least one member selected from the group consisting of α-cyclodextrin, β-cyclodextrin, and γ-cyclodextrin.

13. The production method according to claim 8, wherein the γ-cyclodextrin glucanotransferase is derived from at least one bacterium selected from the group consisting of bacteria belonging to the genus Bacillus, bacteria belonging to the genus Brevibacterium, bacteria belonging to the genus Klebsiella, and bacteria belonging to the genus Corynebacterium.
